(19) United States
(12) Patent Application Publication
(10) Pub. No.: US 2024/0360270 A1
(43) Pub. Date: Oct. 31, 2024

(54) METHOD AND SYSTEM FOR PREPARING POLYURETHANE-BASED SOFT TISSUE BIOADHESIVE

(71) Applicant: GUANGZHOU SOONHEAL MEDICAL TECHNOLOGY CO., LTD, Guangzhou City (CN)

(72) Inventors: Xuetao SHI, Guangzhou City (CN); Chengkai XUAN, Guangzhou City (CN); Wei YANG, Guangzhou City (CN); Kai WU, Beijing City (CN)

(73) Assignee: GUANGZHOU SOONHEAL MEDICAL TECHNOLOGY CO., LTD, Guangzhou City (CN)

(21) Appl. No.: 18/530,284

(22) Filed: Dec. 6, 2023

(30) Foreign Application Priority Data
Apr. 28, 2023 (CN) 2023104762041

(51) Int. Cl. C08G 18/66; A61L 24/04; C08G 18/77; C08G 18/82

(52) U.S. Cl. CPC C08G 18/6674; A61L 24/046; C08G 18/771; C08G 18/82; A61B 17/00491

(57) ABSTRACT

This invention falls within the biomedical glue and intelligent manufacturing technical domain, offering a method and system for preparing a polyurethane-based soft tissue bioadhesive. The method involves obtaining a viscosity measurement value using a viscometer during the acquisition of an aliphatic polyurethane prepolymer crucial for soft tissue bioadhesive preparation. An industrial digital camera captures a vessel image, and a miscellaneous offset sequence is calculated from the image. The adjustment expansion degree is then computed based on the viscosity measurement value and the miscellaneous offset sequence. The reaction process is ultimately controlled in conjunction with the adjustment expansion degree, utilizing a side reaction to observe characteristics caused by impurities through a color change. This approach enables the observation of side reaction characteristics, enhancing the accuracy of predicting the reaction progress endpoint. Synchronous monitoring of multiple reaction vessels optimizes adjustment time, significantly reducing productivity waste and improving production efficiency.

### TECHNICAL FIELD

The present invention belongs to the technical field of biomedical glue and intelligent manufacturing, and particularly relates to a method and system for preparing a polyurethane-based soft tissue bioadhesive.

### BACKGROUND ART

In the synthesis process of preparing aliphatic polyurethane prepolymer, —NCO is consumed by —OH. Therefore, an end point of the reaction is positively correlated with the concentration of —NCO in a system. The method commonly used by those skilled in the art is to locate the end point of the reaction by di-n-butylamine titration. One of the unavoidable problems by using di-n-butylamine titration is that moisture in the air consumes —NCO or oxygen in the air consumes —CNO. Although these problems can be solved by using vacuum and inert gas, there are always insurmountable problems such as insufficient vacuum degree and nitrogen leakage in actual production, which will lead to huge differences in the end time of the same preparation process or reaction process. The uncertainty of time brings quite unfavorable unstable factors to large-scale preparation of a soft tissue bioadhesive. When the reaction process is incomplete, a large number of chemical raw materials will be wasted. The spare time after the complete reaction will bring waste of production energy to large-scale production, and the spare time after the complete reaction will further probably reduce the yield of the aliphatic polyurethane prepolymer, thus reducing economic benefits.

Therefore, if the termination time of the reaction process cannot be dynamically and automatically identified, the large-scale preparation of medical adhesives will not achieve the ideal productivity. In the process of synthesis reaction, viscosity can reflect the progress of the reaction process to a certain extent. However, since the generation speed of products or the reaction rate is constantly changing with the process, there will always be impurities. Therefore, it is impossible to accurately predict the end point of the process. A side reaction of the reaction caused by impurities may be observed through a color change, thus providing hindering quantification of the preparation progress of the aliphatic polyurethane prepolymer and improving the accuracy of determining the end point of the process.

### SUMMARY

An object of the present invention is to provide a method and system for preparing a polyurethane-based soft tissue bioadhesive, so as to solve one or more technical problems existing in the prior art and at least provide a beneficial selection or creation condition.

In order to achieve the above-mentioned object, according to an aspect of the present invention, a method for preparing a polyurethane-based soft tissue bioadhesive is provided. The soft tissue bioadhesive includes a component A and a component B. The component A is an aliphatic polyurethane prepolymer based on long-chain polyethylene glycol and small molecular polyol. The component B is an aliphatic modified secondary amine curing agent. The component A and the component B are mixed according to a molar weight ratio of functional groups —NCO:—NH=1:1 to form the soft tissue bioadhesive.

Further, the method for obtaining the component A includes: reacting long-chain polyethylene glycol with L-lysine diisocyanate to obtain an intermediate, and adding a small molecular polyol chain extender to obtain an aliphatic polyurethane prepolymer. The long-chain polyethylene glycol is long-chain PEG, and the L-lysine diisocyanate is LDI. The molar weight ratio of the functional group —NCO of LDI to the functional group —OH of PEG in the intermediate ranges from 2:1 to 4:1. The molar weight ratio of the functional group —NCO of LDI to the functional group —OH of PEG in the aliphatic polyurethane prepolymer ranges from 1.2:1 to 2:1. The long-chain PEG is composed of one or more of PEG800, PEG1000, PEG1500, and PEG2000. The small molecular polyol chain extender is composed of one or more of glycerol, pentaerythritol, and glucose.

The method for obtaining the component B includes: reacting aliphatic diprimary amine with alpha, beta-unsaturated carbonyl compound according to the molar ratio of functional groups —NH2:—C═C of 1.2:1 under the condition of transition metal catalysis with a mass fraction of 0.1%, and then obtaining an aliphatic modified secondary amine curing agent by column chromatography separation.

Further, the process of obtaining the aliphatic polyurethane prepolymer includes the following steps:
S100: arranging a viscometer in a reaction vessel, and obtaining a viscosity measurement value through the viscometer;
S200: arranging an industrial digital camera, and capturing a vessel image by using the industrial digital camera;
S300: calculating a miscellaneous offset sequence through the vessel image;
S400: calculating an adjustment expansion degree according to the viscosity measurement value and the miscellaneous offset sequence of the reaction vessel;
S500: controlling a reaction process in combination with the adjustment expansion degree.

Further, in step S100, the method for arranging a viscometer in a reaction vessel and obtaining a viscosity measurement value through the viscometer includes: measuring the viscosity of liquid in the reaction vessel in real time by the viscometer which is any one of a Brookfield viscometer, an on-line vibrating viscometer, or a rotary viscometer, and taking a value obtained by the measurement as the viscosity measurement value. A time interval of acquiring the viscosity measurement value is T which ranges from 0.5 s to 2 s.

Further, in step S200, the method for arranging an industrial digital camera and capturing a vessel image by using the industrial digital camera includes: photographing a solution in the reaction vessel by the industrial digital camera which is an industrial CCD camera or a cmos camera, graying the obtained image, identifying and intercepting a solution region in the reaction vessel from the image by an edge detection algorithm, and taking the finally intercepted image as the vessel image. The frequency of acquiring the vessel image is the same as the time interval of acquiring the viscosity measurement value.

Further, in step S300, the method for calculating a miscellaneous offset sequence through the vessel image includes:
arranging gray values of pixels in the vessel image in ascending order to form a first gray sequence, and intercepting a segment from an upper quartile to a lower quartile of the first gray sequence as a second gray sequence;
denoting a mean of the elements in the second gray sequence as egr_sls, and calculating a sub-sinking parameter sd_idx:sd_idx=ceil (60/T), where T is the time interval of acquiring the viscosity measurement value, and ceil( ) is a round-up function;
forming a sequence from egr_sls obtained at each time as a mean gray sequence egr_ls, representing a serial number of the time with i1, representing an i1^{st} element of the mean gray sequence with egr_lsᵢ₁, and calculating an offset parameter ly_idxi1 at the i1^{st} time:

$${ly\_idx}_{i 1} = min \{ egr\_l1 [ i 1 - sd\_idx ) : i 1 ] \} exp ( {egr\_ls}_{i 1} \div {egr\_ls}_{i 1 - 1} ) ,$$

where min{ } is a minimum function, and egr_ls [(i1−sd_idx):i1] represents a set of elements i1−sd_idx to i1 of the mean gray sequence;
taking a sequence composed of offset parameters at each time as an offset sequence, if an element in the offset sequence is larger than a previous element, defining time corresponding to the element as satisfying an offset condition, and acquiring each offset time to form a sequence as the miscellaneous offset sequence.

The problem of insufficient data concentration occurs in the process of screening the miscellaneous offset sequence since the data screening condition is weak and direct in the process of obtaining the result and the phenomenon of periodic high drift of the gray value in the image cannot be accurately located. However, the prior art cannot solve the problem. In order to make the miscellaneous offset sequence more accord with the screening condition, to solve the problem, and to eliminate the phenomenon of weak data screening condition, the present invention provides a more preferred scheme as follows.

Preferably, in step S300, the method for calculating a miscellaneous offset sequence through the vessel image may further include: arranging gray values of pixels in the vessel image in ascending order to form a first gray sequence, and intercepting a segment from an upper quartile to a lower quartile of the first gray sequence as a second gray sequence;
denoting an arithmetic mean of the elements of the second gray sequence as egr_sls, denoting a median of the second gray sequence as mgr_sls, representing the number of elements in the second gray sequence with noi_sls, and calculating a basic gray eigenvalue bs_grsv:

$$bs\_grsv = mgr\_sls \times exp ( \frac{egr\_sls - mgr\_sls}{noi\_sls} ) ;$$

where exp( ) represents an exponential function with a natural constant e as the base;
denoting a difference between mgr_sls at a time and mgr_sls at the previous time as a medium gray difference m_dis, acquiring the medium gray differences of each historical time to form a sequence as a first gray difference sequence, if the values of a plurality of consecutive elements in the first gray difference sequence are all larger than 0 or less than 0, defining that a one-way step event occurs at a plurality of times, taking the number of the plurality of times as a one-way step length of the one-way step event, searching for all one-way step events in the first gray difference sequence, and taking an arithmetic mean of the one-way step lengths of the one-way step events as an auxiliary sinking parameter fd_idx;

$$calculating a sinking parameter sk\_gap : sk\_gap = min \{ fd\_idx , ceil ( 60 / T ) \} ;$$

where T is the time interval of acquiring the viscosity measurement value, and ceil( ) is the round-up function. The minimum value of the first sk_gap gray eigenvalues at the current time is taken as a quasi-gray eigenvalue. A sequence of the quasi-gray eigenvalues at each historical time is constructed as a quasi-gray sequence. If the value of an element in the quasi-gray sequence is larger than that of the previous element, the time is marked as a first offset time. Each first offset time is acquired to form a sequence as the miscellaneous offset sequence.

The beneficial effects are as follows. Since the miscellaneous offset sequence is calculated according to the gray change features in the vessel image, the time and position of an intensified gray change can be accurately marked, and preparation is made for further quantifying the viscosity change trend or quantifying the reaction obstruction degree through the position of the intensified gray change. Therefore, the feature information extraction when the viscosity changes with the reaction process in the process of synthesis reaction can be improved, and the accuracy is improved for further regulating the end point of the reaction.

Further, in step S400, the method for calculating an adjustment expansion degree according to the viscosity measurement value and the miscellaneous offset sequence of the reaction vessel includes:
taking a viscosity residual at one time as a difference between the viscosity measurement values at this time and at the previous time, taking the time in the miscellaneous offset sequence as a miscellaneous time, and acquiring viscosity residuals at each historical time of the reaction vessel to form a sequence as a residual sequence;
defining, if the value of an element in the residual sequence is larger or smaller than that of the previous element and the following element, time corresponding to the element as an auxiliary mark time;
denoting time in the residual sequence, which is the auxiliary mark time and the miscellaneous time, as a first mark point, the number of first mark points in the residual sequence being nomls, and calculating the adjustment expansion degree ct_idx of the reaction vessel as:

$$ct\_idx = {nomls}^{- 1} {\sum }_{i 3 = 1}^{nomls} exp ( \vert \frac{{mls}_{i 3} ( l ) - {e\_mls}_{i 3}}{{ds\_mls}_{i 3} + 1} \vert ) ;$$

where i3 is taken as a serial number of the first mark point, a fragment from the first mark points i3 to i3−1 in the residual sequence is intercepted as mlsᵢ₃, mlsᵢ₃(l) represents the last element in mlsᵢ₃, e_mlsᵢ₃ represents a mean of the elements in mlsᵢ₃, and ds_mlsᵢ₃ is a difference between maximum and minimum values of the elements in mlsᵢ₃.

Since the phenomenon of mark deviation often occurs in the process of calculating the adjustment expansion degree, the problem of data alignment between the mark time and the miscellaneous time will be caused, and the prior art cannot solve the problem of data alignment. In order to better solve the problem and eliminate the phenomenon of mark deviation, the present invention provides a more preferred scheme as follows.

Preferably, in step S400, the method for calculating an adjustment expansion degree according to the viscosity measurement value and the miscellaneous offset sequence of the reaction vessel may further include:
taking a viscosity residual at one time as a difference between the viscosity measurement values at this time and at the previous time, taking the time in the miscellaneous offset sequence as a miscellaneous time, and acquiring viscosity residuals at each historical time of the reaction vessel to form a sequence as a residual sequence;
if the miscellaneous times are consecutive, defining that progress inhibition events occur at the consecutive times, denoting the number of times occupied by the progress inhibition event as an inhibition distance hdds, and acquiring a median of the inhibition distance of each progress inhibition event as an inhibition distance parameter;
if the inhibition distance of the progress inhibition event is larger than or equal to the inhibition distance parameter, taking the first miscellaneous time and the last miscellaneous time of the progress inhibition event as inhibition mark point times, if the inhibition distance of the progress inhibition event is larger than or equal to the inhibition distance parameter, taking the middle time of the progress inhibition event as an inhibition mark point time, and if there are two middle times of the progress inhibition event, selecting time closest to the other inhibition event as an inhibition mark point time, the time closest to the other inhibition event referring to: if the inhibition event closest to the current inhibition event occurs before the current inhibition event, selecting the former, otherwise selecting the latter;
calculating a loss fitness value LssRt at each mark point time:
intercepting a sequence between the current mark point time and the previous mark point time in the intercepted residual sequence as stls;

$$LssRt = ln ( \vert mean ( larger ) / mean ( stls ) \vert ) ;$$

where mean( ) is a mean function, and larger is each value larger than mean(larger) in stls. The adjustment expansion degree ct_idx of the reaction vessel is calculated as:

$$cct\_idx = {\sum }_{i 2 = 1}^{noLss} \frac{{LssRt}_{i 2} \times {rsk}_{i 2}}{noLss} ;$$

where i2 is a cumulative variable, noLss is the number of mark point times, LssRtᵢ₂ represents the loss fitness value of an i2^{nd} mark point time, rskᵢ₂ is an inhibition probability, and the value represented is the ratio of the total number of mark point times to the total number of miscellaneous times between the i2^{nd} mark point time and the current time.

The beneficial effects are as follows. In the reaction process, viscosity can reflect the progress of the reaction process to a certain extent. However, since the generation speed of products or the reaction rate is constantly changing with the process, there will always be problems such as impurities or insufficient void and nitrogen leakage, and it is impossible to accurately predict the end point of the process. A side reaction of the reaction caused by impurities may be observed through a color change, and hindering quantification of the reaction progress can be provided by mutual combination, thus improving the accuracy of predicting the end point of the process.

Further, in step S500, the method for controlling a reaction process in combination with the adjustment expansion degree includes: denoting a default reaction time of a reactant in the reaction vessel as prd;
acquiring adjustment expansion degrees of the reaction vessels to form an expansion sequence;
denoting a mean of maximum and minimum values in the expansion sequence as sepi, and denoting a mean of the expansion sequence as eoi, a total adjustment expansion coefficient being set_rg:set_rg=eoi/sepi, and a total adjustment time being set_len:set_len=prd×set_rg;
starting to record each total adjustment time set_len upon a period of time from the reaction to prd/2, constructing a sequence as an adjustment parameter sequence in which a minimum value is an optimal adjustment time, obtaining an optimal reaction time continuously, and when the reaction time reaches the optimal adjustment time, stopping the reaction process of the reaction vessel.

Further, the aliphatic diprimary amine is composed of one or more of 1,5-pentanediamine, 1,6-hexanediamine, and N′N-bis(3-aminopropyl)methylamine, the alpha, beta-unsaturated carbonyl compound is composed of one or more of methyl acrylate, butyl acrylate, and diethyl maleate, and the transition metal is composed of one or more of ceric ammonium nitrate, yttrium nitrate, cobalt chloride, and ferric chloride.

Preferably, all undefined variables in the present invention, if not explicitly defined, may be manually set thresholds.

The present invention also provides a system for preparing a polyurethane-based soft tissue bioadhesive. The system for preparing a polyurethane-based soft tissue bioadhesive includes: a processor, a memory, and a computer program stored in the memory and executable on the processor. The processor, when executing the computer program, implements the steps in the method for preparing a polyurethane-based soft tissue bioadhesive. The system for preparing a polyurethane-based soft tissue bioadhesive may be operated in a desktop computer, a laptop computer, a palmtop computer, a cloud data center, and other computing devices. The operable system may include, but is not limited to, a processor, a memory, a server cluster. The processor executes the computer program in the following units of the system:
a viscosity collection unit, configured to arrange a viscometer in a reaction vessel, and obtain a viscosity measurement value through the viscometer;
an image capturing unit, configured to arrange an industrial digital camera, and capture a vessel image by using the industrial digital camera;
a miscellaneous quantification unit, configured to calculate a miscellaneous offset sequence through the vessel image;
an adjustment model unit, configured to calculate an adjustment expansion degree according to the viscosity measurement value and the miscellaneous offset sequence of the reaction vessel; and
a dynamic regulation unit, configured to control a reaction process in combination with the adjustment expansion degree.

The present invention has the following beneficial effects. The present invention provides a method and system for preparing a polyurethane-based soft tissue bioadhesive. By using a side reaction of a reaction, the characteristics of the side reaction caused by impurities can be observed through a color change. Therefore, the hindering quantification of the reaction progress can be provided, and the accuracy of an end point of the prediction process can be improved. It is often necessary to operate reaction vessels synchronously in the same batch production process to improve the operation fluency and production efficiency of a preparation line. Therefore, by monitoring the plurality of reaction vessels synchronously, the synchronous adjustment time is optimized, the productivity waste is greatly reduced, and the production efficiency is improved.

### BRIEF DESCRIPTION OF THE DRAWINGS

The above and other features of the present invention will become more apparent from a detailed description of the embodiments illustrated in conjunction with the accompanying drawings in which like reference numerals refer to the same or similar elements. It will be apparent that the drawings described below are only some examples of the present invention. Other drawings may be obtained from these drawings without any creative effort by those of ordinary skill in the art, in which:

FIG. 1 shows a flowchart of a method for preparing a polyurethane-based soft tissue bioadhesive.

FIG. 2 shows a structural diagram of a system for preparing a polyurethane-based soft tissue bioadhesive.

### DETAILED DESCRIPTION OF THE EMBODIMENTS

A clear and complete description of the concept, the specific structure, and the resulting technical effects of the present invention will be given below in conjunction with the examples and the accompanying drawings, in order to fully understand the object, scheme, and effects of the present invention. It is to be noted that the examples in the present application and the features in the examples may be combined with each other without conflict.

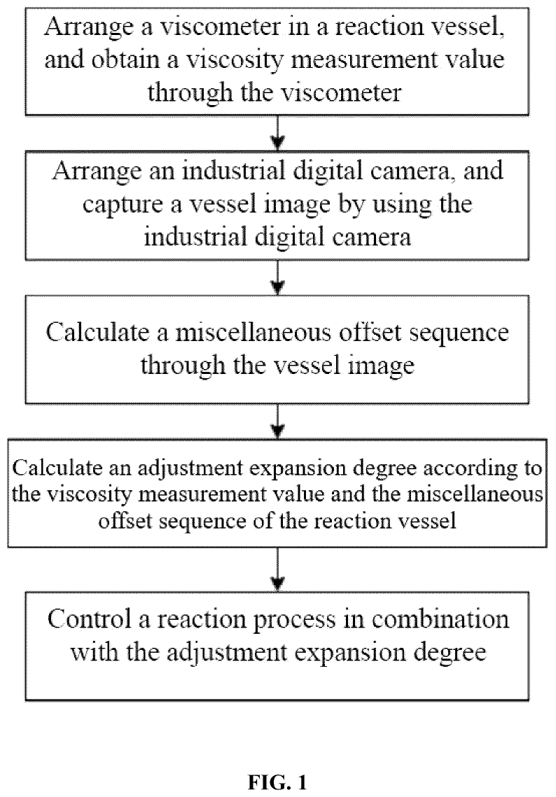

FIG. 1 shows a flowchart of a method for preparing a polyurethane-based soft tissue bioadhesive. The method for preparing a polyurethane-based soft tissue bioadhesive according to embodiments of the present invention will be illustrated below in conjunction with FIG. 1. The method includes the following steps:

### Example 1: The Soft Tissue Bioadhesive Includes a Component A and a Component B

The component A is an aliphatic polyurethane prepolymer based on long-chain polyethylene glycol and small molecular polyol. The component B is an aliphatic modified secondary amine curing agent. The component A and the component B are mixed according to a molar weight ratio of functional groups —NCO:—NH=1:1 to form the soft tissue bioadhesive.

Further, the method for obtaining the component A includes: reacting long-chain polyethylene glycol with L-lysine diisocyanate to obtain an intermediate, and adding a small molecular polyol chain extender to obtain an aliphatic polyurethane prepolymer. The long-chain polyethylene glycol is long-chain PEG, and the L-lysine diisocyanate is LDI. The molar weight ratio of the functional group —NCO of LDI to the functional group —OH of PEG in the intermediate is 2:1. The molar weight ratio of the functional group —NCO of LDI to the functional group —OH of PEG in the aliphatic polyurethane prepolymer is 1.5. The long-chain PEG is PEG1000. The small molecular polyol chain extender is glycerol.

The method for obtaining the component B includes: reacting aliphatic diprimary amine with alpha, beta-unsaturated carbonyl compound according to the molar ratio of functional groups —NH2:—C═C of 1.2:1 under the condition of transition metal catalysis with a mass fraction of 0.1%, and then obtaining an aliphatic modified secondary amine curing agent by column chromatography separation.

Further, the process of obtaining the aliphatic polyurethane prepolymer includes the following steps:
S100: Arrange a viscometer in a reaction vessel, and obtain a viscosity measurement value through the viscometer.
S200: Arrange an industrial digital camera, and capture a vessel image by using the industrial digital camera.
S300: Calculate a miscellaneous offset sequence through the vessel image.
S400: Calculate an adjustment expansion degree according to the viscosity measurement value and the miscellaneous offset sequence of the reaction vessel.
S500: Control a reaction process in combination with the adjustment expansion degree.

Further, in step S100, the method for arranging a viscometer in a reaction vessel and obtaining a viscosity measurement value through the viscometer includes: measuring the viscosity of liquid in the reaction vessel in real time by the viscometer which is an on-line vibrating viscometer, and taking a value obtained by the measurement as the viscosity measurement value. A time interval of acquiring the viscosity measurement value is T. T=1 s.

Further, in step S200, the method for arranging an industrial digital camera and capturing a vessel image by using the industrial digital camera includes: photographing a solution in the reaction vessel by the industrial digital camera which may be an industrial CCD camera, graying the obtained image, identifying and intercepting a solution region in the reaction vessel from the image by an edge detection algorithm, and taking the finally intercepted image as the vessel image. The frequency of acquiring the vessel image is the same as the time interval of acquiring the viscosity measurement value.

Further, in step S300, the method for calculating a miscellaneous offset sequence through the vessel image includes:
arranging gray values of pixels in the vessel image in ascending order to form a first gray sequence, and intercepting a segment from an upper quartile to a lower quartile of the first gray sequence as a second gray sequence;
denoting a mean of the elements in the second gray sequence as egr_sls, and calculating a sub-sinking parameter sd_idx:sd_idx=ceil(60/T);
forming a sequence from egr_sls obtained at each time as a mean gray sequence egr_ls, representing a serial number of the time with i1, representing an i1^{st} element of the mean gray sequence with egr_lsi1, and calculating an offset parameter ly_idxi1 at the i1^{st} time:

$$ly\_idxi 1 = min \{ egr\_ls [ ( i 1 - sd\_idx ) : i 1 ] \} exp ( egr\_lsi 1 \div egr\_lsi 1 - 1 ) ;$$

where min{ } is a minimum function, and egr_ls [(i1−sd_idx):i1] represents a set of elements i1−sd_idx to i1 of the mean gray sequence;
taking a sequence composed of offset parameters at each time as an offset sequence, if an element in the offset sequence is larger than a previous element, defining time corresponding to the element as satisfying an offset condition, and acquiring each offset time to form a sequence as the miscellaneous offset sequence.

Further, in step S400, the method for calculating an adjustment expansion degree according to the viscosity measurement value and the miscellaneous offset sequence of the reaction vessel includes:
taking a viscosity residual at one time as a difference between the viscosity measurement values at this time and at the previous time, taking the time in the miscellaneous offset sequence as a miscellaneous time, and acquiring viscosity residuals at each historical time of the reaction vessel to form a sequence as a residual sequence;
defining, if the value of an element in the residual sequence is larger or smaller than that of the previous element and the following element, time corresponding to the element as an auxiliary mark time;
denoting time in the residual sequence, which is the auxiliary mark time and the miscellaneous time, as a first mark point, the number of first mark points in the residual sequence being nomls, and calculating the adjustment expansion degree ct_idx of the reaction vessel as:

$$ct\_idx = {nomls}^{- 1} {\sum }_{i 3 = 1}^{nomls} exp ( \vert \frac{{mls}_{i 3} ( l ) - {e\_mls}_{i 3}}{{ds\_mls}_{i 3} + 1} \vert )$$

where i3 is taken as a serial number of the first mark point, a fragment from the first mark points i3 to i3−1 in the residual sequence is intercepted as mlsᵢ₃, mlsᵢ₃(l) represents the last element in mlsᵢ₃, e_mlsᵢ₃ represents a mean of the elements in mlsᵢ₃, and ds_mlsᵢ₃ is a difference between maximum and minimum values of the elements in mlsᵢ₃.

Further, in step S500, the method for controlling a reaction process in combination with the adjustment expansion degree includes: denoting a default reaction time of a reactant in the reaction vessel as prd;
acquiring adjustment expansion degrees of the reaction vessels to form an expansion sequence;
denoting a mean of maximum and minimum values in the expansion sequence as sepi, and denoting a mean of the expansion sequence as eoi, a total adjustment expansion coefficient being set_rg:set_rg=eoi/sepi, and a total adjustment time being set_len:set_len=prd×set_rg;
starting to record each total adjustment time set_len upon a period of time from the reaction to prd/2, constructing a sequence as an adjustment parameter sequence in which a minimum value is an optimal adjustment time, obtaining an optimal reaction time continuously, and when the reaction time reaches the optimal adjustment time, stopping the reaction process of the reaction vessel.

Further, the aliphatic diprimary amine is 1,5-pentanediamine. The alpha, beta-unsaturated carbonyl compound is diethyl maleate. The transition metal is ammonium ceric nitrate.

### Example 2

The aliphatic polyurethane prepolymer is prepared by using the method in Example 1. The difference between Example 2 and Example 1 is that the method for calculating a miscellaneous offset sequence through the vessel image includes:
arranging gray values of pixels in the vessel image in ascending order to form a first gray sequence, and intercepting a segment from an upper quartile to a lower quartile of the first gray sequence as a second gray sequence;
denoting an arithmetic mean of the elements of the second gray sequence as egr_sls, denoting a median of the second gray sequence as mgr_sls, representing the number of elements in the second gray sequence with noi_sls, and calculating a basic gray eigenvalue bs_grsv:

$$bs\_grsv = mgr\_sls \times exp ( \frac{egr\_sls - mgr\_sls}{noi\_sls} )$$

where exp( ) represents an exponential function with a natural constant e as the base;
denoting a difference between mgr_sls at a time and mgr_sls at the previous time as a medium gray difference m_dis, acquiring the medium gray differences of each historical time to form a sequence as a first gray difference sequence, if the values of a plurality of consecutive elements in the first gray difference sequence are all larger than 0 or less than 0, defining that a one-way step event occurs at a plurality of times, taking the number of the plurality of times as a one-way step length of the one-way step event, searching for all one-way step events in the first gray difference sequence, and taking an arithmetic mean of the one-way step lengths of the one-way step events as an auxiliary sinking parameter fd_idx;
calculating a sinking parameter sk_gap:sk-gap=min{fd_idx, ceil(60/T)};
where T is the time interval of acquiring the viscosity measurement value, and ceil( ) is the round-up function. The minimum value of the first sk_gap gray eigenvalues at the current time is taken as a quasi-gray eigenvalue. A sequence of the quasi-gray eigenvalues at each historical time is constructed as a quasi-gray sequence. If the value of an element in the quasi-gray sequence is larger than that of the previous element, the time is marked as a first offset time. Each first offset time is acquired to form a sequence as the miscellaneous offset sequence.

The difference between Example 2 and Example 1 is that the method for calculating an adjustment expansion degree includes:
taking a viscosity residual at one time as a difference between the viscosity measurement values at this time and at the previous time, taking the time in the miscellaneous offset sequence as a miscellaneous time, and acquiring viscosity residuals at each historical time of the reaction vessel to form a sequence as a residual sequence;
if the miscellaneous times are consecutive, defining that progress inhibition events occur at the consecutive times, denoting the number of times occupied by the progress inhibition event as an inhibition distance hdds, and acquiring a median of the inhibition distance of each progress inhibition event as an inhibition distance parameter;
if the inhibition distance of the progress inhibition event is larger than or equal to the inhibition distance parameter, taking the first miscellaneous time and the last miscellaneous time of the progress inhibition event as inhibition mark point times, if the inhibition distance of the progress inhibition event is larger than or equal to the inhibition distance parameter, taking the middle time of the progress inhibition event as an inhibition mark point time, and if there are two middle times of the progress inhibition event, selecting time closest to the other inhibition event as an inhibition mark point time (the time closest to the other inhibition event referring to: if the inhibition event closest to the current inhibition event occurs before the current inhibition event, selecting the former, otherwise selecting the latter);
calculating a loss fitness value LssRt at each mark point time:
intercepting a sequence between the current mark point time and the previous mark point time in the intercepted residual sequence as stls;

$$LssRt = ln ( \vert mean ( larger ) / mean ( stls ) \vert ) ;$$

where mean( ) is a mean function, and larger is each value larger than mean(larger) in stls. The adjustment expansion degree ct_idx of the reaction vessel is calculated as:

$$ct\_idx = {\sum }_{i 2 = 1}^{noLss} \frac{{LssRt}_{i 2} \times {rsk}_{i 2}}{noLss}$$

where i2 is a cumulative variable, noLss is the number of mark point times, LssRtᵢ₂ represents the loss fitness value of an i2^{nd} mark point time, rskᵢ₂ is an inhibition probability, and the value represented is the ratio of the total number of mark point times to the total number of miscellaneous times between the i2^{nd} mark point time and the current time.

### Comparative Example 1

The aliphatic polyurethane prepolymer is prepared by using the method in Example 1. The difference between Comparative Example 1 and Example 1 is that an adjustment time in the preparation of the aliphatic polyurethane prepolymer is determined by using a conventional —NCO group detection method, specifically including:
determining the adjustment time in the preparation of the aliphatic polyurethane prepolymer by the —NCO group (end point criterion) detection method:
C_Step1: Prepare 0.1 mol/L of di-n-butylamine/toluene solution, 0.1 mol/L of hydrochloric acid aqueous solution (standard solution), and 1 mass percent of bromophenol blue/ethanol indicator.
C_Step2: Weigh 0.1 g of prepolymer sample, put the sample in a 250 ml of conical flask (samples are not added in a blank group), add 25 ml of di-n-butylamine/toluene solution, and stir for 15 minutes to make it completely react.
C_Step3: Add 50 ml of isopropanol and 3-4 drops of bromophenol blue indicator, slowly drop the hydrochloric acid standard solution with an acid burette, stop dropping after the color of the solution changes from dark blue to yellow green and keeps unchanged for 1 minute, and record the volume of the consumed hydrochloric acid standard solution.
C_Step4: Calculate —NCO concentration (mol/l) as: NCO=(V0−V)*C/m, where V0 is the volume of the hydrochloric acid standard solution consumed by the blank group, V is the volume of the hydrochloric acid standard solution consumed by the experimental group, C is the concentration of the hydrochloric acid standard solution, and m is the mass of prepolymer.
C_Step5: Adjust the preparation process of the aliphatic polyurethane prepolymer when the color of the solution in C_Step3 changes from purple blue to yellow green and the value of the —NCO concentration (mol/l) reaches 50% of an initial value.

TABLE 1
Comparison Results of Examples 1 to 2 and Comparative Example 1

| | Comparative | Example | Example |
|---|---|---|---|
| Items | Example | 1 | 2 |
| Inspection time (mins) | 30 min | 0 | 0 |
| Inspection frequency (number) | 5 | — | — |
| Total inspection time (mins) | 120 | — | — |
| Complete preparation time (hour) | 7 | 5 | 4.5 |
| Yield (%) | 90 | 92 | 93.5 |

The inspection time is the operation time for determining the reaction end point of the process of preparing the aliphatic polyurethane prepolymer in the comparative example. The inspection frequency is the number of determinations for reaction termination in the preparation process. The total inspection time is the time from the beginning to the end of the operation to determine the reaction termination. The complete preparation time is the time from the beginning to the end of the preparation process of the aliphatic polyurethane prepolymer (including inspecting determination of the reaction termination). From the table, it can be seen that the preparation methods in Example 1 and Example 2 have great advantages in terms of production efficiency and yield compared with the conventional adjustment process using the conventional —NCO group detection method.

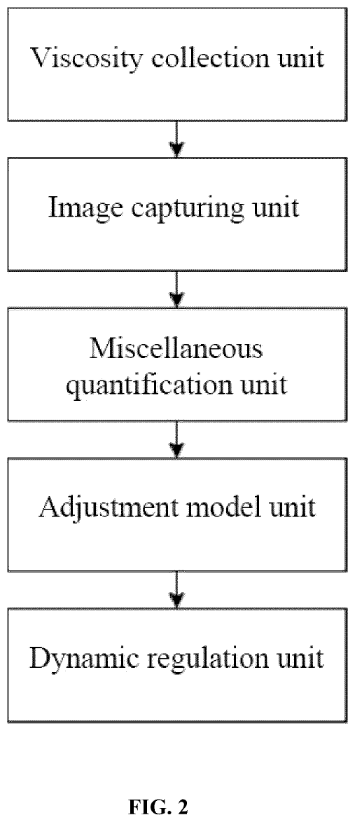

An example of the present invention provides a system for preparing a polyurethane-based soft tissue bioadhesive. FIG. 2 shows a structural diagram of a system for preparing a polyurethane-based soft tissue bioadhesive. The system for preparing a polyurethane-based soft tissue bioadhesive according to this example includes: a processor, a memory, and a computer program stored in the memory and executable on the processor. The processor, when executing the computer program, implements the steps in the example of the system for preparing a polyurethane-based soft tissue bioadhesive.

The system includes: a memory, a processor, and a computer program stored in the memory and executable on the processor. The processor executes the computer program in the following units of the system:
a viscosity collection unit, configured to arrange a viscometer in a reaction vessel, and obtain a viscosity measurement value through the viscometer;
an image capturing unit, configured to arrange an industrial digital camera, and capture a vessel image by using the industrial digital camera;
a miscellaneous quantification unit, configured to calculate a miscellaneous offset sequence through the vessel image;
an adjustment model unit, configured to calculate an adjustment expansion degree according to the viscosity measurement value and the miscellaneous offset sequence of the reaction vessel; and
a dynamic regulation unit, configured to control a reaction process in combination with the adjustment expansion degree.

The system for preparing a polyurethane-based soft tissue bioadhesive may be operated in a desktop computer, a laptop computer, a palmtop computer, a cloud server, and other computing devices. The system for preparing a polyurethane-based soft tissue bioadhesive may include, but is not limited to, the processor and the memory. Those skilled in the art will appreciate that the example is merely an example of the system for preparing a polyurethane-based soft tissue bioadhesive and does not constitute a limitation to the system for preparing a polyurethane-based soft tissue bioadhesive. The system may include more or less components, or a combination of certain components, or different components. For example, the system for preparing a polyurethane-based soft tissue bioadhesive may also include an input/output device, a network access device, a bus, and the like.

The processor may be a Central Processing Unit (CPU), other general purpose processors, a Digital Signal Processor (DSP), an Application Specific Integrated Circuit (ASIC), a Field-Programmable Gate Array (FPGA) or other programmable logic devices, discrete gates or transistor logic devices, discrete hardware components, and the like. The general purpose processor may be a microprocessor or the processor may be any conventional processor. The processor is a control center of an operating system of the system for preparing a polyurethane-based soft tissue bioadhesive, and various interfaces and lines are used to connect various parts of an operable system of the system for preparing a polyurethane-based soft tissue bioadhesive.

The memory may be configured to store the computer program and/or modules, and the processor implements various functions of the system for preparing a polyurethane-based soft tissue bioadhesive by running or executing the computer program and/or modules stored in the memory and invoking data stored in the memory. The memory may mainly include a stored program area and a stored data area. The stored program area may store an operating system, an application required by at least one function (such as a sound playback function and an image playback function), and the like. The stored data area may store data (such as audio data and phone books) created according to the use of a mobile phone. Furthermore, the memory may include a high-speed random access memory, and may also include a non-volatile memory, such as a hard disk, a memory, a plug-in hard disk, a Smart Media Card (SMC), a Secure Digital (SD) card, a flash card, at least one disk storage device, a flash memory device, or another volatile solid-state storage device.

Although the description of the present invention has been fairly exhaustive and several examples have been specifically described, it is not intended to be limited to any of these details or examples or any particular example, thereby effectively covering the predetermined scope of the present invention. Furthermore, the above description of the present invention with examples foreseeable by the inventor is intended to provide a useful description, while non-substantial modifications to the present invention that are not currently foreseen may still represent equivalent modifications to the present invention.

What is claimed is:

1. A method for preparing a polyurethane-based soft tissue bioadhesive, the soft tissue bioadhesive comprising a component A and a component B, wherein:
the component A is an aliphatic polyurethane prepolymer based on long-chain polyethylene glycol and small molecular polyol, and the component B is an aliphatic modified secondary amine curing agent, and the component A and the component B are mixed according to a molar weight ratio of functional groups —NCO:—NH=1:1 to form the soft tissue bioadhesive;
the method for obtaining the component A comprises: reacting the long-chain polyethylene glycol with L-lysine diisocyanate to obtain an intermediate, and adding the small molecular polyol chain extender to obtain the aliphatic polyurethane prepolymer, the long-chain polyethylene glycol being long-chain PEG, the L-lysine diisocyanate being LDI, the molar weight ratio of the functional group —NCO of LDI to the functional group —OH of PEG in the intermediate ranging from 2:1 to 4:1, the molar weight ratio of the functional group —NCO of LDI to the functional group —OH of PEG in the aliphatic polyurethane prepolymer ranging from 1.2:1 to 2:1, the long-chain PEG being composed of one or more of PEG800, PEG1000, PEG1500, and PEG2000, and the small molecular polyol chain extender being composed of one or more of glycerol, pentaerythritol, and glucose;
the method for obtaining the component B comprises: reacting aliphatic diprimary amine with alpha, beta-unsaturated carbonyl compound according to the molar ratio of functional groups —NH2:—C═C of 1.2:1 under the condition of transition metal catalysis with a mass fraction of 0.1%, and then obtaining the aliphatic modified secondary amine curing agent by column chromatography separation; and
the process of obtaining the aliphatic polyurethane prepolymer comprises the following steps:
S100: arranging a viscometer in a reaction vessel, and obtaining a viscosity measurement value through the viscometer;
S200: arranging an industrial digital camera, and capturing a vessel image by using the industrial digital camera;
S300: calculating a miscellaneous offset sequence through the vessel image;
S400: calculating an adjustment expansion degree according to the viscosity measurement value and the miscellaneous offset sequence of a reaction vessel; and
S500: controlling a reaction process in combination with the adjustment expansion degree,
wherein
in step S300, the method for calculating a miscellaneous offset sequence through the vessel image comprises:
arranging gray values of pixels in the vessel image in ascending order to form a first gray sequence, and intercepting a segment from an upper quartile to a lower quartile of the first gray sequence as a second gray sequence;
denoting a mean of the elements in the second gray sequence as egr_sls, and calculating a sub-sinking parameter sd_idx:sd_idx=ceil (60/T);
forming a sequence from egr_sls obtained at each time as a mean gray sequence egr_ls, representing a serial number of the time with i1, representing an i1^{st} element of the mean gray sequence with egr_lsᵢ₁, and calculating an offset parameter ly_idxᵢ₁ at the i1^{st} time: ly_idx i1=min{egr_ls[(i1−sd_idx):i1]}exp(egr_lsi1÷egr_lsi1−1), wherein min{ } is a minimum function, and egr_ls [(i1−sd_idx):i1] represents a set of elements i1−sd_idx to i1 of the mean gray sequence; and
taking a sequence composed of offset parameters at each time as an offset sequence, if an element in the offset sequence is larger than a previous element, defining time corresponding to the element as satisfying an offset condition, and acquiring each offset time to form a sequence as the miscellaneous offset sequence;
in step S400, the method for calculating an adjustment expansion degree according to the viscosity measurement value and the miscellaneous offset sequence of the reaction vessel comprises:
taking a viscosity residual at one time as a difference between the viscosity measurement values at this time and at the previous time, taking the time in the miscellaneous offset sequence as a miscellaneous time, and acquiring viscosity residuals at each historical time of the reaction vessel to form a sequence as a residual sequence;
defining, if the value of an element in the residual sequence is larger or smaller than that of the previous element and the following element, time corresponding to the element as an auxiliary mark time; and
denoting time in the residual sequence, which is the auxiliary mark time and the miscellaneous time, as a first mark point, the number of first mark points in the residual sequence being nomls, and calculating the adjustment expansion degree ct_idx of the reaction vessel as:
$$ct\_idx = {nomls}^{- 1} {\sum }_{i 3 = 1}^{nomls} exp ( \vert \frac{{mls}_{i 3} ( l ) - {e\_mls}_{i 3}}{{ds\_mls}_{i 3} + 1} \vert ) [ [ ; ] ] ,$$
wherein i3 is taken as a serial number of the first mark point, a fragment from the first mark points i3 to i3−1 in the residual sequence is intercepted as mlsᵢ₃, mlsᵢ₃(l) represents the last element in mlsᵢ₃, e_mlsᵢ₃ represents a mean of the elements in mlsᵢ₃, and ds_mlsᵢ₃ is a difference between maximum and minimum values of the elements in mlsᵢ₃; and
in step S500, the method for controlling a reaction process in combination with the adjustment expansion degree comprises:
denoting a default reaction time of a reactant in the reaction vessel as prd;
acquiring adjustment expansion degrees of the reaction vessels to form an expansion sequence, denoting a mean of maximum and minimum values in the expansion sequence as sepi, and denoting a mean of the expansion sequence as eoi, a total adjustment expansion coefficient being set_rg:set_rg=eoi/sepi, and a total adjustment time being set_len: set_len=prd×set_rg; and
starting to record each total adjustment time set_len upon a period of time from the reaction to prd/2, constructing a sequence as an adjustment parameter sequence in which a minimum value is an optimal adjustment time, obtaining an optimal reaction time continuously, and when the reaction time reaches the optimal adjustment time, stopping the reaction process of the reaction vessel.

2. The method for preparing the polyurethane-based soft tissue bioadhesive according to claim 1, wherein in step S100, the method for arranging the viscometer in the reaction vessel and obtaining the viscosity measurement value through the viscometer comprises:
measuring the viscosity of liquid in the reaction vessel in real time by the viscometer which is any one of an on-line vibrating viscometer or a rotary viscometer, and taking a value obtained by the measurement as the viscosity measurement value, a time interval of acquiring the viscosity measurement value being T which ranges from 0.5 s to 2 s.

3. The method for preparing the polyurethane-based soft tissue bioadhesive according to claim 1, wherein in step S200, the method for arranging the industrial digital camera and capturing the vessel image by using the industrial digital camera comprises: photographing a solution in the reaction vessel by the industrial digital camera which is an industrial CCD camera or a cmos camera, graying the obtained image, identifying and intercepting a solution region in the reaction vessel from the image by an edge detection algorithm, and taking the finally intercepted image as the vessel image, the frequency of acquiring the vessel image being the same as the time interval of acquiring the viscosity measurement value

4. The method for preparing the polyurethane-based soft tissue bioadhesive according to claim 1, wherein the aliphatic diprimary amine is composed of one or more of 1,5-pentanediamine, 1,6-hexanediamine, and N′N-bis(3-aminopropyl)methylamine, the alpha, beta-unsaturated carbonyl compound is composed of one or more of methyl acrylate, butyl acrylate, and diethyl maleate, and the transition metal is composed of one or more of ceric ammonium nitrate, yttrium nitrate, cobalt chloride, and ferric chloride.

5. A system for preparing the polyurethane-based soft tissue bioadhesive, comprising: a processor, a memory, and a computer program stored in the memory and executable on the processor, wherein the processor, when executing the computer program, implements the steps in the method for preparing the polyurethane-based soft tissue bioadhesive according to claim 1, and the system for preparing the polyurethane-based soft tissue bioadhesive is operated in a desktop computer, a laptop computer, a palmtop computer, a cloud data center, and other computing devices.
